(19) United States
(12) Patent Application Publication
(10) Pub. No.: US 2025/0387139 A1
(43) Pub. Date: Dec. 25, 2025

(54) VARIABLE EXPOSURE NEEDLE

(71) Applicant: BOSTON SCIENTIFIC SCIMED, INC., Maple Grove, MN (US)

(72) Inventors: Colby HARRIS, Norfolk, MA (US); Ryan V. WALES, Shrewsbury, MA (US)

(21) Appl. No.: 19/209,137

(22) Filed: May 15, 2025

Related U.S. Application Data
(60) Provisional application No. 63/663,504, filed on Jun. 24, 2024.

(51) Int. Cl. A61B 17/34; A61B 1/012; A61B 17/00; A61B 17/30

(52) U.S. Cl. CPC A61B 17/3494; A61B 1/012; A61B 17/3478; A61B 2017/00862; A61B 2017/306

(57) ABSTRACT

A device controlling a penetration depth of an endoscopic instrument includes a member and an effector. The member includes a proximal portion extending from a proximal end to a distal portion having an open distal end. The distal portion is received within a working channel of an insertion instrument in Insertion Configuration. The distal portion flares laterally outward from the proximal portion so that Diameter of the open distal end is greater than Diameter of the proximal portion. The member moves the distal portion between Insertion Configuration and Operative Configuration in which the distal portion extends distally beyond a distal end of the channel. The effector moves between Retracted Configuration in which a distal end of the effector is received within the member and Extended Configuration in which the effector is moved distally beyond the distal end of the member to penetrate tissue to a desired depth.

### PRIORITY CLAIM

The present disclosure claims priority to U.S. Provisional Patent Application Ser. No. 63/663,504 filed Jun. 24, 2024; the disclosure of which is incorporated herewith by reference.

### FIELD

The present disclosure relates to an endoscopic device for controlling a depth of penetration of a needle into tissue for the injection of fluids between tissue layers.

### BACKGROUND

Endoscopic procedures often utilize injections of substances for various therapeutic applications. For example, endoscopic mucosal resection (EMR) and endoscopic submucosal dissection (ESD) involve the injection of fluids between the muscularis and mucosal layers to elevate a selected portion of mucosal tissue relative to surrounding areas of tissue. This may be used to facilitate, for example, resection of mucosal and/or submucosal tissue while minimizing the risk that the entire thickness of tissue will be penetrated during the resection (e.g., opening the interior of the body lumen to the peritoneum). However, there is a risk that the needle used in the injection (e.g., during the EMR or EDS procedure) will penetrate the entire thickness of tissue and enter the peritoneum minimizing or eliminating the effectiveness of any injection while raising the risk of complications such as infection.

### SUMMARY

The present disclosure relates to a device for controlling a depth of penetration of an endoscopic instrument into a target portion of tissue within a living body. The device includes an elongate hollow member sized and shaped to be slidably received within a working channel of an insertion instrument. The elongate hollow member includes a proximal portion extending from a proximal end which, during use, remains outside the body accessible to a user to a distal portion having an open distal end. The distal portion is configured to be received within the working channel in an insertion configuration. The distal portion flares laterally outward from the proximal portion so that a diameter of the open distal end is greater than a diameter of the proximal portion. The elongate hollow member is movable longitudinally within the working channel to move the distal portion between the insertion configuration and an operative configuration in which the distal portion extends distally beyond a distal end of the working channel.

The device also includes an end effector slidably received within the elongate hollow member for movement between a retracted configuration in which a distal end of the end effector is received within the elongate hollow member and an extended configuration in which the end effector is moved distally beyond the distal end of the elongate hollow member to penetrate a target portion of tissue to a desired depth.

In an embodiment, the end effector includes a needle configured to inject fluid into the target portion of tissue.

In an embodiment, an outer diameter of the distal end of the elongate hollow member is selected to be smaller than an inner diameter of the working channel of the insertion instrument with which the device is to be used.

In an embodiment, the distal portion is biased to expand to the operative configuration when the distal portion is advanced out of the working channel of the insertion instrument.

In an embodiment, the distal portion is formed of a flexible biocompatible polymer.

In an embodiment, the distal portion is formed of silicone.

In an embodiment, the entire elongate hollow member is formed of the flexible biocompatible polymer.

In an embodiment, in the operative configuration, the diameter of the distal end of the elongate hollow member is greater than an inner diameter of the working channel.

In an embodiment, the distal portion includes a plurality of rib members extending longitudinally therethrough to increase a column strength of the distal portion to a level selected to ensure that the distal portion does not collapse due to the pressure exerted thereon by the target portion of tissue drawn against the distal portion through suction applied through the elongate hollow member.

In an embodiment, the elongate hollow member includes an annular seal projecting radially outward from an outer surface thereof at a location proximal of the distal portion, the annular seal being configured to engage an inner wall of the working channel to prevent the flow past the annular seal of gases within an annular space between the inner wall of the working channel and the elongate hollow member.

In an embodiment, the elongate hollow member includes an opening through a wall thereof proximal of the annular seal, the opening permitting fluid communication between a lumen of the elongate hollow member and the annular space between the inner wall of the working channel and the elongate hollow member.

In an embodiment, the proximal portion of the elongate hollow member includes an opening to a lumen of the elongate hollow member.

In an embodiment, the end effector is a needle configured to be passed through the elongate hollow member into the target portion of tissue drawn into the distal end of the elongate hollow member.

In addition, the present disclosure related to a system for controlling a depth of penetration of an endoscopic instrument into a target portion of tissue within a living body. The device includes an insertion instrument and an elongate hollow member sized and shaped to be slidably received within a working channel of the insertion instrument. The elongate hollow member includes a proximal portion extending from a proximal end which, during use, remains outside the body accessible to a user to a distal portion having an open distal end. The distal portion is configured to be received within the working channel in an insertion configuration.

The distal portion flares laterally outward from the proximal portion so that a diameter of the open distal end is greater than a diameter of the proximal portion. The elongate hollow member is movable longitudinally within the working channel to move the distal portion between the insertion configuration and an operative configuration in which the distal portion extends distally beyond a distal end of the working channel.

In addition, the device includes an end effector slidably received within the elongate hollow member for movement between a retracted configuration in which a distal end of the end effector is received within the elongate hollow member and an extended configuration in which the end effector is moved distally beyond the distal end of the elongate hollow member to penetrate a target portion of tissue to a desired depth.

In an embodiment, the elongate hollow member includes an annular seal projecting radially outward from an outer surface thereof at a location proximal of the distal portion, the annular seal being configured to engage an inner wall of the working channel to prevent the flow past the annular seal of gases within an annular space between the inner wall of the working channel and the elongate hollow member.

In addition, the present disclosure relates to a method for controlling a depth of penetration of an endoscopic instrument into a target portion of tissue within a living body. The method includes inserting into a working channel of an insertion instrument, an elongate hollow member; moving the elongate hollow member distally through the working channel until an increased diameter distal end of a distal portion thereof moves distally out of the working channel; applying suction through a lumen of the elongate hollow member to draw a selected portion of tissue into the distal end of the elongate hollow member; and inserting an end effector slidably through the elongate hollow member into the selected portion of tissue to penetrate a target portion of tissue to a desired depth.

In an embodiment, the end effector includes a needle configured to inject fluid into the selected portion of tissue.

In an embodiment, the distal portion is biased to expand to an operative configuration when the distal portion is advanced out of the working channel of the insertion instrument.

In an embodiment, the elongate hollow member includes an annular seal projecting radially outward from an outer surface thereof at a location proximal of the distal portion, the annular seal being configured to engage an inner wall of the working channel to prevent the flow past the annular seal of gases within an annular space between the inner wall of the working channel and the elongate hollow member, the method further comprising applying negative pressure to a proximal portion of the annular space between the inner wall of the working channel and the elongate hollow member.

In an embodiment, the elongate hollow member includes an opening through a wall thereof proximal of the annular seal, the opening permitting fluid communication between the lumen of the elongate hollow member and the annular space between the inner wall of the working channel and the elongate hollow member so that the negative pressure introduced into the annular space between the inner wall of the working channel and the elongate hollow member passes into the lumen of the elongate hollow member to draw the selected portion of tissue into the distal portion of the elongate hollow member.

### BRIEF DESCRIPTION

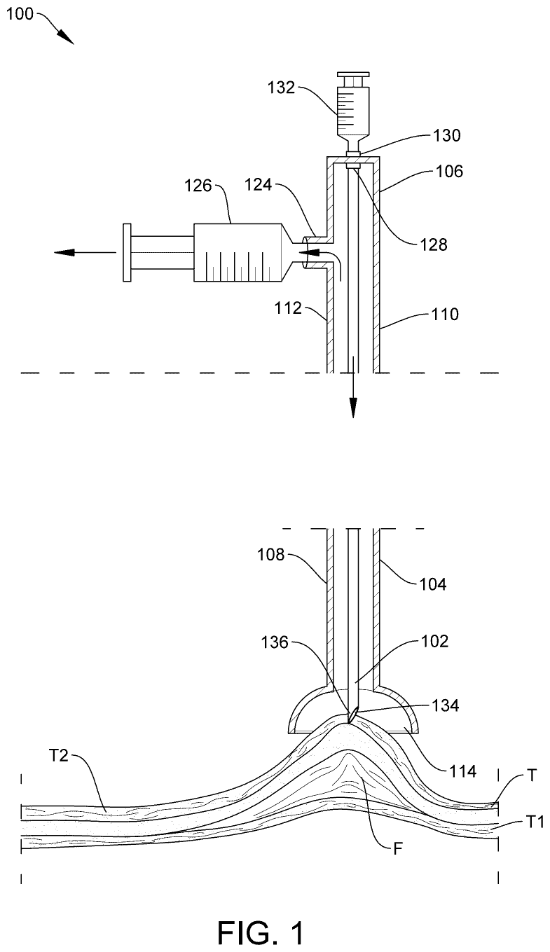

FIG. 1 shows a cross-sectional view of a system according to an exemplary embodiment of the present disclosure;

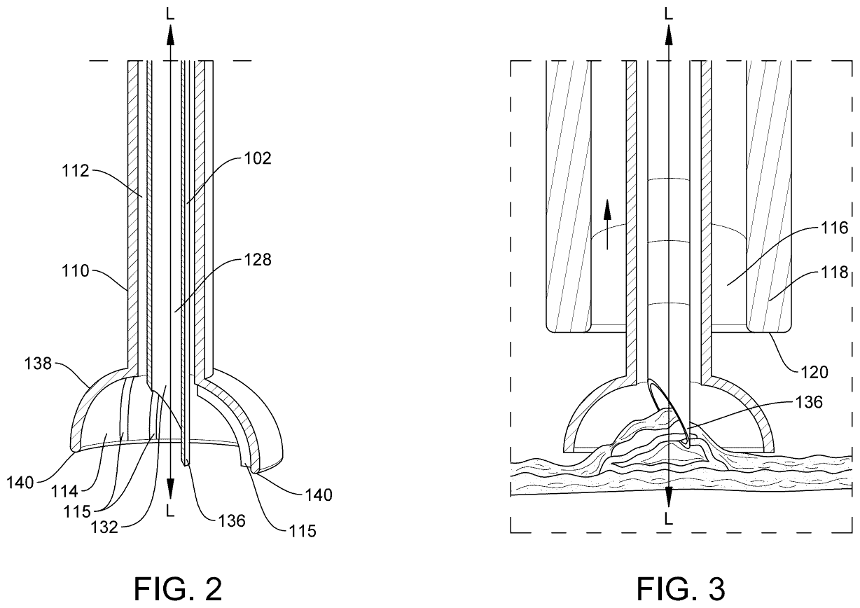

FIG. 2 shows a perspective, cross-sectional view of the distal portion of the system of FIG. 1;

FIG. 3 shows a cross-sectional view of the distal portion of the system of FIG. 1 with a needle inserted into a target portion of tissue;

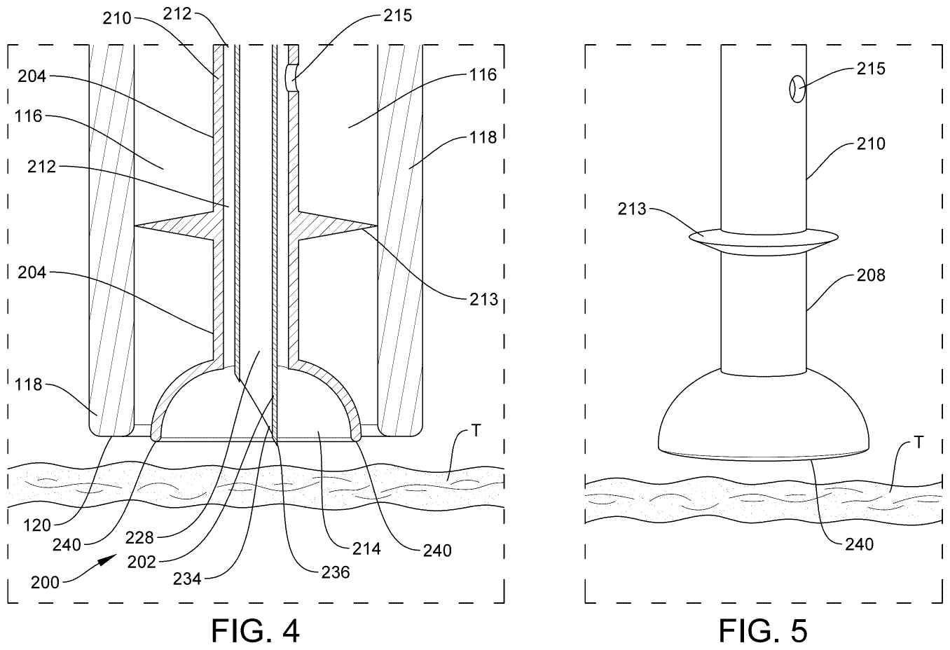

FIG. 4 shows a cross-sectional view of a distal portion of a device according to a further embodiment; and

FIG. 5 shows a perspective view of the distal portion of the device of FIG. 4.

### DETAILED DESCRIPTION

The present disclosure may be further understood with reference to the following description and appended drawings, wherein like elements are referred to with the same reference numerals. The present disclosure relates to an endoscopic needle system and, in particular, relates to a device for controlling a depth of penetration of an endoscopic needle into a target portion of tissue into which a therapeutic agent is to be injected.

It should be noted that although the exemplary embodiments specifically describe a needle system for EMR and ESD, it will be understood by those of skill in the art that the exemplary system may be utilized for any of variety endoscopic procedures in which a needle is to be inserted into a target portion of tissue to a desired depth. It should also be noted that the terms “proximal” and “distal,” as used herein, are intended to refer to a direction toward (proximal) and away from (distal) a user of the device (e.g., physician).

As shown in FIGS. 1-3, an endoscopic system 100 for insertion of a needle 102 into a target portion of tissue T in, for example, the wall of an organ of the digestive system, includes a insertion device 104 extending from a proximal portion 106 which, during use, remains outside the body accessible to a user to a distal portion 108 that is configured to be inserted into the body to a location adjacent to the target portion of tissue T to be treated. For example, the treatment may involve an injection therapy application in which a fluid is injected into the target tissue T to elevate a mucosal layer of tissue relative to portions of tissue surrounding the target tissue T. As would be understood by those skilled in the art, such treatments (e.g., EMR and ESD) may be employed to facilitate the resection of mucosal tissue while minimizing the risk that the entire thickness of the tissue T will be penetrated. In such cases, fluids F may be injected between muscularis T₁ and mucosal T₂ tissue layers to form a bump or raised portion of the mucosal layer T₂ in the area adjacent to the injection.

The insertion device 104 includes a flexible elongate member 110 that defines a lumen 112 extending therethrough to an open distal end 114. The needle 102 is a flexible needle formed, as would be understood by those skilled in the art, of a material such as stainless steel, nitinol, biocompatible polymer, etc. that is sufficiently flexible as to be able to be passed through the working channel 116 of a flexible insertion instrument such as a flexible endoscope 118 when, for example, the endoscope 118 is inserted into a living body along a tortuous path to a location adjacent to the target portion of tissue T. For example, the endoscope 118 may be inserted through a natural bodily orifice such as the mouth into the digestive tract (e.g., through the esophagus into the stomach and from there into the small intestine) to a location adjacent to the target portion of tissue T.

The elongate member 110 is then inserted through the working channel 116 to exit the distal end 120 of the endoscope 118 to engage the target tissue T as desired. Alternatively, the elongate member may be loaded into the working channel 116 before the endoscope 118 is inserted into the body. For example, the proximal end of the elongate member 110 may be inserted into the working channel 116 via an opening in the distal end 120 and slid through the endoscope 118 until the distal end 114 is received within the working channel 116. As will be described in more detail below, the distal end 114 of the elongate member 110 of this embodiment has an outer diameter less than an inner diameter of the working channel 116 so that the distal end 114 may be received in the working channel 116 in an uncompressed state.

Alternatively, the distal end 114 may be formed of a material naturally biased to an expanded operative configuration but which may be compressed and/or folded into the working channel 116 in a reduced diameter insertion configuration. When the endoscope 118 has reached a target location adjacent to tissue to be treated, the elongate member 110 may be advanced distally until the distal end 114 extends out of the working channel 116. As the distal end 114 of this alternate embodiment moves out of the working channel 116 and is no longer constrained thereby, the distal end 114 expands radially outward into its operative configuration (i.e., with the distal end 114 now having an outer diameter greater than the inner diameter of the working channel 116). Those skilled in the art will understand that the distal end 114 (and/or the entire elongate member 110) may be formed, for example, of silicone or a flexible biocompatible polymer shaped to bias the distal end 114 toward its operative configuration so that, as it leaves the working channel 116 while the member is configured to permit it to be re-compressed as the distal end 114 is withdrawn proximally into the working channel 116 (e.g., after completion of the procedure).

The distal end 114 may also optionally include one or more rib members 115 extending longitudinally therethrough to enhance its column strength (on a proximal to distal axis) to ensure the distal end does not flex (collapse) under the force applied by the tissue to the distal end as the suction is applied to draw the tissue into the distal end 114. After the procedure has been completed and the suction is no longer applied, the operator may pull the elongate member 110 proximally into the working channel so that contact with the distal end of the endoscope collapses the distal end 114 which is then withdrawn proximally into the working channel 116.

The proximal portion 106 of the insertion device 104 includes a needle port 122 including an opening sized and shaped to receive the needle 102 therethrough and an injection port 124 configured to engage a source of fluid pressure which may, for example, include a syringe 126 or any other suitable known source of fluid pressure. As will be made clear below, negative pressure is to be applied within the lumen 112 of the elongate member 110. Thus, the needle port 122 of this embodiment is configured to seal around the needle 102 so that negative pressure introduced into the lumen 112 does not leak out of the elongate member 110 at the needle port 122.

The needle 102 is hollow defining a lumen 128 extending therethrough from a proximal opening 130 in the proximal end of the needle 102 to an opening 134 in a tissue piercing distal end 136 of the needle 102. The needle port 122 is configured to couple to a source of fluid 132 so that fluids from the source of fluid 132 pass into the lumen 128 of the needle and through the needle 102 to be injected into the target tissue T into which the distal end 136 of the needle 102 has been inserted. Alternatively, the proximal end of the needle 102 may be coupled directly to the source of fluid 132 so that the fluids can be introduced directly into the lumen 128.

The elongate member 110 defines a longitudinal axis L and has a distal end 138 with a bell-shaped configuration. That is, the distal end 138 of the elongate member 110 flares outward relative to portions of the elongate member 110 proximal thereto so that a diameter of the distal end 138 in a plane transverse to the axis L is greater than the diameter of the more proximal portions of the elongate member 110. As indicated above, in this embodiment, the distal end 138 has an outer diameter at its distal end (its maximum diameter) slightly smaller than an inner diameter of the working channel 116 with which the device is to be used. Thus, the elongate member 110 may be slidably inserted through the working channel of the endoscope (e.g., by passing the proximal end of the elongate member 110 into an opening in the distal end of the endoscope and threading the elongate member 110 through the endoscope until it reaches the proximal end of the endoscope 118.

The distal rim 140 of the elongate member 110 is configured to sealingly engage tissue surrounding the target tissue T so that negative pressure introduced into the lumen 112 (e.g., via operation of the syringe 126) creates suction that draws the tissue T proximally into the distal end 138 while holding surrounding portions of tissue in place. As would be understood by those skilled in the art, due to the relative rigidity of the mucularis layer T₁ as compared to the mucosal layer T₂ when suction is applied through the lumen 112, the mucosal layer T₂ is drawn proximally while the mucularis layer T₁ is held generally in position so that a separation between these layers is increased.

This increases the space into which the distal end 136 needle 102 may then be positioned for the injection of fluids into the target tissue T. That is, the suction creates a temporary bulge in the intestinal wall to facilitate the proper placement of the distal end 136 of the needle 102 by increasing the depth of the space into which the distal end 136 of the needle 102 may be inserted, decreasing the risk that the needle 102 will be passed through the full thickness of the tissue—i.e., enabling a user of the system 100 to more easily place the distal end 136 of the needle 102 as desired while the sharp distal end of the needle 102 remains further from the outer surface of the intestinal wall. When the needle has been placed as desired (e.g., into the mucosal layer T₂ distal of the epithelium and proximal of the mucosal layer T₁), the user operates the source of fluid 132 to inject fluid into this location via the lumen 128 to enhance and make more lasting the bulge created temporarily by the suction in the lumen 112. As would be understood by those skilled in the art, his facilitates the resection of tissue from the epithelium and mucosal layers while minimizing the risk that the muscularis layer T₁ will be pierced.

FIGS. 4 and 5 show a system 200 according to a further embodiment in which the proximal portion of the system (not shown) is constructed substantially similarly to the proximal portion 106 of the system 100. The system 200 includes an insertion device 204 extending from a proximal portion (not shown) which, during use, remains outside the body accessible to a user to a distal portion 208 configured to be inserted into the body to a location adjacent to the target portion of tissue T to be treated. Although, this embodiment will also describe an application involving injection therapy, those skilled in the art will understand that the system 200 may be used for any application in which it is desired to treat or mark for treatment any portion of tissue after creating a separation between layers of tissue in, for example, the wall of the body lumen.

For example, the needle 202 of the system 200 may be replaced or supplemented by a device for the application of one or more markers (e.g., radiopaque markers) embedded in the tissue T to facilitate the identification of the marked tissue for a later procedure (e.g., resection, ablation, etc.). In this embodiment, as with the system 100 fluids F are injected between muscularis and mucosal tissue layers to form a bump or raised portion of the mucosal layer in the area adjacent to the injection. The insertion device 204 includes a flexible elongate member 210 that defines a lumen 212 extending therethrough to an open distal end 214. The needle 202 is a flexible needle formed, as would be understood by those skilled in the art, of a material such as stainless steel, nitinol, biocompatible polymer, etc. that is sufficiently flexible as to be able to be passed through the working channel 116 of the endoscope 118 when the endoscope 118 is inserted into a living body along a tortuous path. The elongate member 210 is then inserted through the working channel 116 to exit the distal end 120 of the endoscope 118 to engage the target tissue T as desired.

Alternatively, the elongate member 210 may be loaded into the working channel 116 before the endoscope 118 is inserted into the body. For example, the proximal end of the elongate member 210 may be inserted into the working channel 116 via an opening in the distal end 120 and slid through the endoscope 118 until the distal end 214 is received within the working channel 116. Similarly to the system 100, the distal end 214 of the elongate member 210 of this embodiment has an outer diameter less than an inner diameter of the working channel 116 so that the distal end 214 may be received in the working channel 116 in an uncompressed state.

Alternatively, the distal end 214 may be formed of a material naturally biased to an expanded operative configuration but which may be compressed and/or folded into the working channel 116 in a reduced diameter insertion configuration. When the endoscope 118 has reached a target location adjacent to tissue to be treated, the elongate member 210 may be advanced distally until the distal end 214 extends out of the working channel 116. As the distal end 214 of this alternate embodiment moves out of the working channel 116 and is no longer constrained thereby, the distal end 214 expands radially outward into its operative configuration (i.e., with the distal end 214 now having an outer diameter greater than the inner diameter of the working channel 116).

The proximal portion (not shown) of the insertion device 204 can be substantially similar to the proximal part except that the proximal portion of the elongate member 210 is configured to pass through the port at the proximal end of the working channel 116. As would be understood by those skilled in the art, the port at the proximal end of the working channel of an endoscope is generally configured to seal around a device inserted into the working channel via the port. This permits suction applied within the working channel to be sealed within the working channel so that the suction is applied at the distal end of the endoscope via the distal opening of the working channel.

Thus, this embodiment includes a distal seal 213 projecting outward from the outer surface of the elongate member 210 to sealingly engage an inner surface of the working channel 116 as well as a suction opening 215 extending through the wall of the elongate member 210 proximal to the seal 213 to open the lumen 212 of the needle 202 to the lumen of the working channel 116. Thus, when the elongate member 210 is inserted into the working channel 116 via the seal at the proximal port to the working channel and suction is applied to the working channel 116, the suction is channeled through the working channel to the seal 213 which prevents the suction from drawing fluids/gases proximally past the seal 213 so that the suction passes into the lumen 212 of the elongate member 210 and is applied to tissue surrounded by the distal end 214 of the elongate member 210 to draw tissue proximally into the distal end 214 as described above in regard to the system 100.

The needle 202 is hollow defining a lumen 228 that extends therethrough from a proximal opening (not shown) in the proximal end of the needle 202 to an opening 234 in a tissue piercing distal end 236 of the needle 202. As described above in regard to the system 100, the needle port of this embodiment may be configured to be coupled to a source of fluid ((not shown) so that fluids from the source pass into the lumen 228 of the needle 202 and through the needle 202 to be injected into the target tissue T into which the distal end 236 of the needle 202 has been inserted. Alternatively, the proximal end of the needle 202 may be coupled directly to the source of fluid so that the fluids can be introduced directly into the lumen 228.

As described above in regard to the system 100, the elongate member 210 defines a longitudinal axis L and has a distal end 238 with a bell-shaped configuration. That is, the distal end 238 of the elongate member 210 flares outward relative to portions of the elongate member 210 proximal thereto so that a diameter of the distal end 238 in a plane transverse to the axis L is greater that the diameter of the more proximal portions of the elongate member 210. As indicated above, in this embodiment, the distal end 238 has an outer diameter at its distal end (its maximum diameter) slightly smaller than an inner diameter of the working channel 116 with which the device is to be used. Thus, the elongate member 210 may be slidably inserted through the working channel of the endoscope (e.g., by passing the proximal end of the elongate member 210 into an opening in the distal end of the endoscope and threading the elongate member 210 through the endoscope 118 until it reaches the proximal end of the endoscope 118).

The distal rim 240 of the elongate member 210 is configured to sealingly engage tissue surrounding the target tissue T so that negative pressure introduced into the lumen 212 creates suction that draws the tissue T proximally into the distal end 238 while holding surrounding portions of tissue T in place. As described above, due to the relative rigidity of the mucularis layer as compared to the mucosal layer when suction is applied through the lumen 212, the mucosal layer is drawn proximally while the mucularis layer is held generally in position so that a separation between these layers is increased—i.e., the intestinal wall in this immediate area is slightly thickened. This increases the space into which the distal end 236 needle 202 may then be positioned for the injection of fluids into the target tissue T. That is, the suction creates a temporary bulge in the intestinal wall to facilitate the proper placement of the distal end 236 of the needle 202 by increasing the depth of the space into which the distal end 236 of the needle 202 may be inserted without passing through the full thickness of the tissue—i.e., enabling a user of the system 200 to more easily place the distal end 236 of the needle 202 as desired while the distal end 236 of the needle 202 remains further from the outer surface of the intestinal wall.

When the needle 202 has been placed as desired (e.g., into the mucosal layer distal of the epithelium and proximal of the mucosal layer), the user operates the source of fluid to inject fluid into this location via the lumen 228 to enhance and make more lasting the bulge created temporarily by the suction in the lumen 212. As would be understood by those skilled in the art, this facilitates the resection of tissue from the epithelium and mucosal layers while minimizing the risk that the muscularis layer will be pierced. For example, the device may permit the needle to move distally into the tissue to a depth of 0.2 mm which would ensure that the needle could not penetrate even the thinnest full thickness portion of the intestinal wall (e.g., 3 to 5 mm thick).

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-15. (canceled)

16. A device for controlling a depth of penetration of an endoscopic instrument into a target portion of tissue within a living body, comprising:
an elongate hollow member sized and shaped to be slidably received within a working channel of an insertion instrument, the elongate hollow member including a proximal portion extending from a proximal end which, during use, remains outside the body accessible to a user to a distal portion having an open distal end, the distal portion being configured to be received within the working channel in an insertion configuration, wherein the distal portion flares laterally outward from the proximal portion so that a diameter of the open distal end is greater than a diameter of the proximal portion, the elongate hollow member being movable longitudinally within the working channel to move the distal portion between the insertion configuration and an operative configuration in which the distal portion extends distally beyond a distal end of the working channel; and
an end effector slidably received within the elongate hollow member for movement between a retracted configuration in which a distal end of the end effector is received within the elongate hollow member and an extended configuration in which the end effector is moved distally beyond the distal end of the elongate hollow member to penetrate a target portion of tissue to a desired depth.

17. The device of claim 16, wherein the end effector includes a needle configured to inject fluid into the target portion of tissue.

18. The device of claim 16, wherein an outer diameter of the distal end of the elongate hollow member is selected to be smaller than an inner diameter of the working channel of the insertion instrument with which the device is to be used.

19. The device of claim 16, wherein the distal portion is biased to expand to the operative configuration when the distal portion is advanced out of the working channel of the insertion instrument.

20. The device of claim 19, wherein the distal portion is formed of a flexible biocompatible polymer.

21. The device of claim 20, wherein the distal portion is formed of silicone.

22. The device of claim 20, wherein the elongate hollow member is formed of the flexible biocompatible polymer.

23. The device of claim 19, wherein, in the operative configuration, the diameter of the distal end of the elongate hollow member is greater than an inner diameter of the working channel.

24. The device of claim 19, wherein the distal portion includes a plurality of rib members extending longitudinally therethrough to increase a column strength of the distal portion to a level selected to ensure that the distal portion does not collapse due to a pressure exerted thereon by the target portion of tissue drawn against the distal portion through suction applied through the elongate hollow member.

25. The device of claim 16, wherein the elongate hollow member includes an annular seal projecting radially outward from an outer surface thereof at a location proximal of the distal portion, the annular seal being configured to engage an inner wall of the working channel to prevent a flow past the annular seal of gases within an annular space between the inner wall of the working channel and the elongate hollow member.

26. The device of claim 25, wherein the elongate hollow member includes an opening through a wall thereof proximal of the annular seal, the opening permitting fluid communication between a lumen of the elongate hollow member and the annular space between the inner wall of the working channel and the elongate hollow member.

27. The device of claim 16, wherein the proximal portion of the elongate hollow member includes an opening to a lumen of the elongate hollow member.

28. The device of claim 16, wherein the end effector is a needle configured to be passed through the elongate hollow member into the target portion of tissue drawn into the distal end of the elongate hollow member.

29. A system for controlling a depth of penetration of an endoscopic instrument into a target portion of tissue within a living body, comprising:
an insertion instrument;
an elongate hollow member sized and shaped to be slidably received within a working channel of the insertion instrument, the elongate hollow member including a proximal portion extending from a proximal end which, during use, remains outside the body accessible to a user to a distal portion having an open distal end, the distal portion being configured to be received within the working channel in an insertion configuration, wherein the distal portion flares laterally outward from the proximal portion so that a diameter of the open distal end is greater than a diameter of the proximal portion, the elongate hollow member being movable longitudinally within the working channel to move the distal portion between the insertion configuration and an operative configuration in which the distal portion extends distally beyond a distal end of the working channel; and
an end effector slidably received within the elongate hollow member for movement between a retracted configuration in which a distal end of the end effector is received within the elongate hollow member and an extended configuration in which the end effector is moved distally beyond the distal end of the elongate hollow member to penetrate a target portion of tissue to a desired depth.

30. The system of claim 29, wherein the elongate hollow member includes an annular seal projecting radially outward from an outer surface thereof at a location proximal of the distal portion, the annular seal being configured to engage an inner wall of the working channel to prevent a flow past the annular seal of gases within an annular space between the inner wall of the working channel and the elongate hollow member.

31. A method for controlling a depth of penetration of an endoscopic instrument into a target portion of tissue within a living body, comprising:
inserting into a working channel of an insertion instrument, an elongate hollow member;
moving the elongate hollow member distally through the working channel until an increased diameter distal end of a distal portion thereof moves distally out of the working channel;
applying suction through a lumen of the elongate hollow member to draw a selected portion of tissue into the distal end of the elongate hollow member; and
inserting an end effector slidably through the elongate hollow member into the selected portion of tissue to penetrate a target portion of tissue to a desired depth.

32. The method of claim 31, wherein the end effector includes a needle configured to inject fluid into the selected portion of tissue.

33. The method of claim 31, wherein the distal portion is biased to expand to an operative configuration when the distal portion is advanced out of the working channel of the insertion instrument.

34. The method of claim 31, wherein the elongate hollow member includes an annular seal projecting radially outward from an outer surface thereof at a location proximal of the distal portion, the annular seal being configured to engage an inner wall of the working channel to prevent a flow past the annular seal of gases within an annular space between the inner wall of the working channel and the elongate hollow member, the method further comprising applying negative pressure to a proximal portion of the annular space between the inner wall of the working channel and the elongate hollow member.

35. The method of claim 34, wherein the elongate hollow member includes an opening through a wall thereof proximal of the annular seal, the opening permitting fluid communication between the lumen of the elongate hollow member and the annular space between the inner wall of the working channel and the elongate hollow member so that the negative pressure introduced into the annular space between the inner wall of the working channel and the elongate hollow member passes into the lumen of the elongate hollow member to draw the selected portion of tissue into the distal portion of the elongate hollow member.
